(19) United States
(12) Patent Application Publication
(10) Pub. No.: US 2007/0055294 A1
(43) Pub. Date: Mar. 8, 2007

(54) Magnetic needle positioner

(76) Inventor: Brandon Giap, San Diego, CA (US)

Correspondence Address: DONN K. HARMS;PATENT & TRADEMARK LAW CENTER, SUITE 100, 12702 VIA CORTINA, DEL MAR, CA, 92014, US

(21) Appl. No.: 11/210,407

(22) Filed: Aug. 23, 2005

(51) Int. Cl. A61B 17/04

(52) U.S. Cl. 606/148

(57) ABSTRACT

A device and method for maintaining a curved sewing needle in an upright position when the needle is deposited thereon during suturing or sewing. The device features a base with a top surface on which a curved needle may be removably deposited. Once deposited upon the top surface of the base, a magnetic field of sufficient force and vector holds the needle to a substantially vertical position wherein it is easier to grasp by a tool or fingers for the next suture. Coaxing the needle to the upright position from a sideways deposit on the top surface can be provided by a sufficient field strength and assisted by dimensioning the top surface of the base to mechanically assist the magnetic force on the deposited needle and also by employing a magnetized needle.

### FIELD OF THE INVENTION

The present invention relates to surgical instruments used when closing an incision or wound in body tissue and to instruments in general that are used for the purpose of sewing fabric or suturing patients. In particular, the present invention relates to an instrument that magnetically assists the positioning of a needle when released from the grip of a user or surgeon. The device provides a means to pre-position the needle for easy grasping subsequent to disengagement from a tool or fingers. Likely applications include conventional surgery, minimally invasive surgical procedures such as laparoscopic and other developing image guided endoscopic techniques that are performed within the body in areas of limited access, and general sewing using a curved needle such as in upholstery or carpeting.

### BACKGROUND OF THE INVENTION

Surgery has been long in development and the relentless advance in techniques and equipment has led to the miracles of our more modern era. Every day thousands of surgeries are performed in the United States alone. Some are emergencies, others elective, but all require the suturing or sewing together of the wounded organs in both repair and closing procedures.

Today, most surgeries are performed by opening up the body cavity through the various layers of muscle and fat. Subsequent to the repair, removal or replacement of the involved organ, this suturing process is an elemental component of the surgical process. Suturing of the wound is a fundamental skill learned early in the career of a surgeon and each develops his own style. Throughout the suturing procedure the surgeon has to release and re-grasp the needle a number of times. Each time the needle is re-grasped, the surgeon must ensure that the needle is positioned and oriented correctly within the jaws of the instrument holding the needle so that the needle can then be re-inserted into and through the tissue to make another stitch.

Accordingly, the surgeon must first determine the position and orientation of the needle in the jaws. This is difficult because the surgeon's view of the needle and the instrument is not always unobstructed, and in the case of non-invasive surgery is via a two-dimensional image transmitted by the camera. Next, the needle may need to be adjusted within the jaws, as necessary, which is difficult because the jaws only open and close.

Further, the surgeon must ensure that the needle is not dropped since the needle is likely to be difficult to locate within the cavity in the person or in the transmitted image. Once located, the re-acquisition of a dropped needle, particularly with the suturing instrument, is a challenge. Often a special instrument and another hand to manipulate the instrument are required, especially in non-invasive surgical applications.

Naturally, failure to locate the needle could pose fatal consequences for the patient and litigation for the physicians. The adequate control of the needle(s) during and after the suturing process noted above is a key issue addressed during surgery. Recently since the 1990's new medical endoscopic procedures have been developed which do not require the body part being repaired to be completely opened and these procedures are being performed with ever increasing frequency.

These minimally invasive medical procedures are defined as those that are carried out by entering the body through the skin or through a body cavity or anatomical opening with the smallest damage possible to these structures. Generally, these procedures also involve the extensive use of remote imaging devices that are utilized by both the primary and attending physicians and staff to coordinate their efforts during the operation. Because the entry wounds that are used to access the surgery area are very small, the cutting and separating of various tissue layers to expose intra-abdominal organs is not required.

One of the most useful benefits from this type of procedure is that patient rehabilitation periods are considerably shorter in comparison with traditional fully invasive surgery. The importance of needle control in this two-dimensional operating environment cannot be over emphasized with regard to both loss and prevention of inadvertent “sticking.”

As has been previously indicated, all of these procedures, be they invasive or non-invasive, require a plurality of incisions which will require later repair through suturing. In practical surgical applications, any simplification of the basic suturing process, especially that of control of the needle, is desirable and is beneficial to the recovery of the patient. Today's cost conscious environment also places a priority on minimal implementation costs in materials and re-training. Much effort has been spent toward achieving those goals and as shall be seen related prior art has examples that propose in several ways to resolve some or all of these issues. None is perfectly satisfactory with regard to results.

### DESCRIPTION OF PRIOR ART

U.S. Pat. No 5,201,744 (Jones) describes a method and device for suturing using a rod with a needle holder. The primary goal of this device is providing a surgical knot tying tool that is useful in surgery procedures for centering a suture knot on a wound in body tissue of limited accessibility and that can be manipulated from an area of accessibility outside the body. The device contains and controls the suture thread and needle and is used in conjunction with other tools to position the suture knots during surgery. While in one embodiment the device employs a magnetized component to retain the needle internally, implementation of the methodology requires the presence of another function specific surgical tool in the surgeon's inventory.

U.S. Pat. No. 5,417,701 (Holmes) defines a suturing instrument that uses a magnet to properly position, orient and hold a suturing needle within its jaws. The magnet is embedded in one of the jaws and when the jaws are near the needle, the magnet attracts the needle to it, causing the needle to move to a predetermined position on the jaw containing the magnet. Numerous mechanical internal components grasp, release and position the needle as required. Specialty needles may also be indicated as noted in the proposal. Beyond the additional expense likely to result from manufacture of this contraption, general use would require that another, procedure-specific tool, be readily available to the operator and tool specific training is implied.

U.S. Pat. No. 5,431,670 (Holmes) depicts a suturing instrument that uses an electromagnet to position, orient and hold a needle at the end of an elongated barrel, thereby eliminating the mechanical jaws which are otherwise required. As indicated in the description, this elegant solution to the simple problem of controlling the positioning of the suture needle involves switches, power supplies, electromagnets, multiple controls, special needle holders, and potentially, specialty suture needles with special coatings. Only the largest hospitals could likely afford to provide this complex device which as described would require significant additional training of the physician, calibration and maintenance procedures.

U.S. Pat. Application No. 2003/0105474 (Bonutti) portrays a mechanism and method intended to provide the capability of passing a medical implement through tissue with magnetic or electromagnetic forces. Several versions are envisioned some with multiple magnets and multiple required instruments. Though this invention eliminates the need to accurately position a separate standard suture needle, it does necessitate development of new as yet to be developed surgical technique and requires a specialized device in addition to those that might normally be found in, say, third world countries.

With respect to the above description, before explaining at least one preferred embodiment of the invention in detail, it is to be understood that the invention is not limited in its application to the details of construction and to the arrangement of the components or steps set forth in the following description or illustrated in the drawings. The various apparatus and methods of the invention are capable of other embodiments and of being practiced and carried out in various ways which will be obvious to those skilled in the art once they review this disclosure. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other devices, methods and systems for carrying out the several purposes of the present disclosed device. It is important, therefore, that the objects and claims be regarded as including such equivalent construction and methodology insofar as they do not depart from the spirit and scope of the present invention.

Further objectives of this invention will be brought out in the following part of the specification, wherein detailed description is for the purpose of fully disclosing the invention without placing limitations thereon.

### SUMMARY OF THE INVENTION

There exists in medicine today a need for a simple method and mechanism to effect control and positioning of the suture needle during surgery that is inexpensive, does not require specialized equipment other than that which might be present in a nominally equipped hospital surgery, and does not imply additional training and specialization on the part of the physician.

It is thus the object of the present invention to provide a simple to use, inexpensive device that is readily incorporated into a previously learned surgical procedure such as suturing and that provides substantially improved control of the surgical needle during and after the process. An additional object of the present invention is to provide enhanced protection against needle sticks. These and other objects are accomplished by a method and apparatus for controlling and manipulating the position of surgical needles during surgery through magnetic interaction with the invented device.

The disclosed device employs a permanent magnet sufficiently powerful for the task at hand, which can be employed in various possible configurations and materials. In a particularly preferred embodiment of the device a toroid or “donut” shaped magnet imbedded within or below the stand or base would be used to attract the suture needle into a final resting in an upright position upon the top surface of the device. Using the magnetic field from the magnet, a needle dropped onto the top surface moves in such a way that the needle starts upright with the needle tip on the top surface and exposing the proximal end upright at preferably approximately one third (⅓) or more of the total length.

In all embodiments of the device disclosed herein and anticipated, a magnet or means to generate a magnetic field is positioned on a base to thereby generate a magnetic field through the top surface of the base. This magnetic field is sufficient to hold a needle deposited on the top surface upright when released from a tool or the hand in cases of a curved or ramped top surface. In the case of a flat top surface the magnetic field is sufficient to urge a needle deposited on the top surface upward and hold it in an upright position.

In physics, a magnetic field is an entity produced by moving electric charges (electric currents) that exerts a force on other moving charges. The quantum-mechanical spin of a particle produces magnetic fields and is acted on by them as though it were a current; this accounts for the fields produced by “permanent” ferro magnets.

A magnetic field is a vector which associates with every point in space, a vector that may vary in time. The direction of the field is the equilibrium direction of a compass needle or other ferrous member placed in the field. In mathematics a vector field is a construction in vector calculus which associates a vector to every point in a Euclidean space. Vector fields are often used in physics to model, for example, the speed and direction of a moving fluid throughout space, or the strength and direction of some force, such as the magnetic or gravitational force, as it changes from point to point. Like the electric field, the magnetic field can be defined by the force it produces. The magnetic force exerted on a moving charge takes the form of a vector product or force vector.
F_{magnetic}=q\vec{v}×\vec{B}=qvB sin θ

The right hand rule is a useful mnemonic for visualizing the direction of a magnetic force as given by the Lorentz force law that calculates the force vector of a moving charge in the equation:

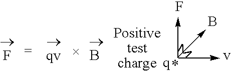

As calculated, the force vector is thus perpendicular to both the velocity v of the charge q and the magnetic field B. The direction of the force is more easily understood as what is conventionally termed in physics as the “right hand rule”. The force relationship above is in the form of a vector product or the force vector of a given magnetic field.

In addition to exerting a force vector which imparts force and direction on ferrous material placed in the field, the magnetic field can can cause a piece of magnetic material placed in the field, to rotate. Consequently, an object which possesses a magnetic dipole moment such as a compass needle or in the device herein, a curved sewing needle, will rotate in response to a magnetic field. Such rotation is ascribed to the effect to a magnetic torque. For a given field the strength of the magnetic torque on an object placed therein is taken as a measure of the magnetic moment of the object.

Using these principles to explain the function of the device herein which was discovered by experimentation and numerous configurations, a curved sewing needle placed in a generally upright position on top of a deport surface of a base can be held in a position substantially normal to the top surface of the base by the force vector of the magnetic field properly generated above the surface. The magnetic field generated above the top surface of the base must be such that the force vector of the field generally aligns with the plane through both ends and the center section of a curved needle and thus exerts the force to hold it upright and normal to the top surface.

Further, it has been found through experimentation that if the needle is magnetized itself, the resulting magnetic torque will cause a curved needle to actually rotate to an upright position if placed on the surface at an angle or parallel to the surface. Still further, it has been found that the needle may be provided magnetized, or once left in the magnetic field generated above the top surface of the base for a short period of time, it will become magnitzed to provide this utility to the user who may simply drop the needle on the top surface at any angle and have it rotate to a vertical position.

The device features an upper surface which may be planar or may have the curvature of the suture needle or may be dimensioned with a depression to mechanically aid positioning and rotation of the deposited needle. The surface of the device may also be coated for sterility or manufactured of an inert biologic or magnetic material or some permutation of all. Preferably the resting surface for the deposited needle is made of material that has a low coefficient of friction to allow the needle to move freely.

The device itself and/or the landing area for the needle is not limited in shape to a circle but can be rectangular, oval, curved or angled or any shape that suits the purpose and anticipated area of use. In the more useful embodiments of the device, the final resting place of the needle on the top surface of the device is preceded by a “landing area” into which the needle is dropped when released from the needle-holding tool or the fingers. The falling needle is then drawn to the magnetic field above the top surface traveling in a pre-defined path. This path has a gradual curve or slant and a needle guide on the sides of the path. The combination of the magnetic field, the force vector and or magnetic torque, act to influence the needle along with the congruence of the gradually curved or slanted path and the embedded needle guide. The end result is that a needle deposited generally upright in the simplest embodiment of the device will be held substantially normal to the top surface of the base. In modes of the device with one or both of magnetized needles or shaped top surfaces to mechanically aid positioning of the needle the result is the needle assuming an upright position no matter at what angle it lands on the top surface of the base. Even more dimensioning of the top surface to move the needle as it is acted on by gravity and the magnetic field will cause it to stand on its distal end next to the tip which leaves the proximal end upright and exposed to be easily re-grasped by the needle holder in a “loading position.” If configured correctly, the top surface of the base acting in concert with the magnetic field will position the needle such that at least ⅓ of the needle is elevated above the top surface for gripping by a tool or the hand.

An object of this invention is to provide a device which pre-positions a needle for stitching and suturing to be easily grasped by a tool or fingers.

Another object of this invention is to provide such a needle positioning device which employs a magnetic force to coax a needle deposited thereon to maintain a generally upright position for grasping.

An additional object of this invention is the provision of such a needle positioning device which will coax a deposited needle upright when deposited on a planar top surface using magnetic force or a combination of magnetic force and mechanical urging.

A still further object of this invention is the provision of such a needle positioning device which additionally dimensions the shape of the top surface to combine with and aid the magnetic field coaxing a needle deposited thereon to an upright position.

Yet another object of this invention is the provision of such a needle positioning device that is sufficiently flexible to be rolled or otherwise compressed through a small opening and subsequently returned to full size for use.

Another object of this invention is the provision of such a flexible needle positioner that may be used in surgery inside of the patient's body and inserted and removed through a very small opening.

These together with other objects and advantages which become subsequently apparent reside in the details of the construction and operation as more fully hereinafter described and claimed, reference being had to the accompanying drawings forming a part thereof, wherein like numerals refer to like parts throughout.

### BRIEF DESCRIPTION OF DRAWING FIGURES AND ITEMS

FIG. 1 depicts a side view of a preferred embodiment of the device 10 in use showing the major components.

FIGS. 2-2a-d depict top and cross sectional views of possible embodiments of the device 10.

FIG. 3 illustrates a top view of the device with the needle in the landing area.

FIG. 4 is a view of the magnetic field formed to coerce the needle upright using a toroid shaped or “donut” magnet.

FIGS. 5-5a are views of a flexible embodiment of the device which may be rolled or folded for insertion into a cavity.

FIG. 6 depicts an arrangement of a plurality of magnets in varying pole positions calculated to generate a larger magnetic field adapted to hold a needle upright.

FIG. 7 shows a top perspective view of another mode of the device having a V shaped ramp depending from a top surface which employs the rams to urge the needle upright.

FIG. 8 is an end view of FIG. 6 showing the ramp descending to a low point and a magnet adjacent to the low point.

FIG. 9 shows a side view of FIG. 8 depicting the needle upright and in position for gripping

### DETAILED DESCRIPTION OF PREFERRED EMBODIMENTS OF THE DISCLOSED DEVICE

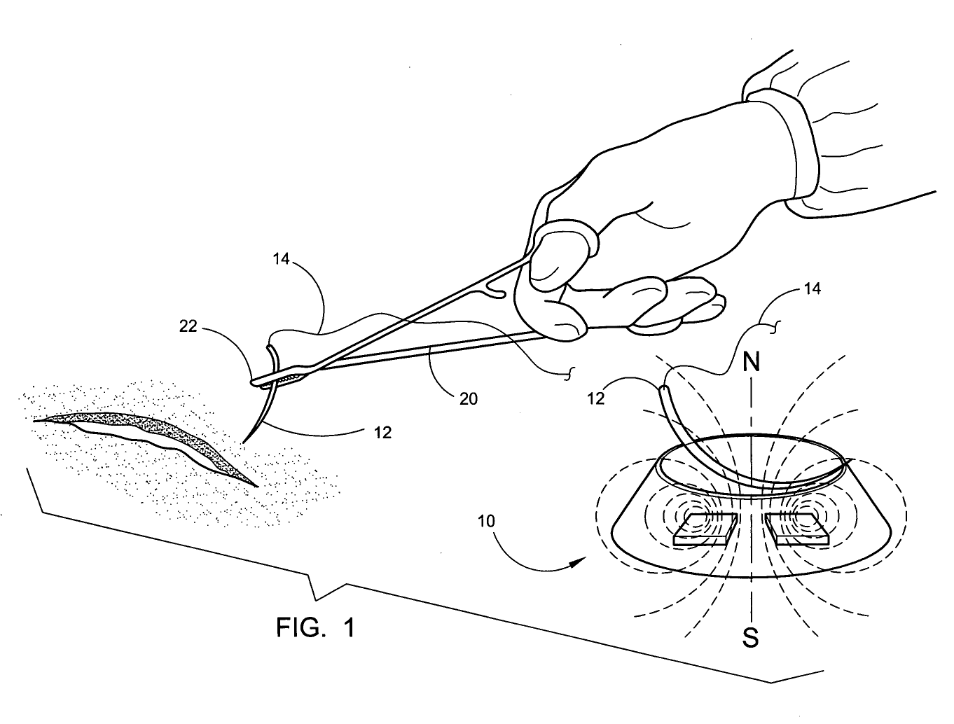

Referring now to the drawings, FIGS. 1-9 disclose some preferred embodiments of the present invention in current preferred modes. The device 10 is depicted in FIG. 1 which displays a side perspective view of a preferred embodiment of the device 10. As shown, the needle 12 is engaged with the suture or thread 14 and is being held firmly in place for use by the tension generated by pressure between the jaws of the gripping end of a needle holder 22. Also shown is the needle 12 upright on the base 18 of the device in the “ready ” position where it is held in a substantially upright position relative to the top surface 15 of the base 18 by a means for generation of a magnetic field 30 above the top surface such as permanent magnets 16. The magnets 16 project the magnetic field 30 in a fashion calculated to hold the magnetically attractive needle 12 substantially upright and in place. The magnetic fields of the device and the top surface of the base may also be employed to preferably coax the needle 12 to a substantially upright position from any position non-perpendicular to the top surface 15 of the device 10 when the needle 12 is released from a grip by the hands or needle holder 22 and dropped on the top surface 15.

Such magnetic fields are produced by electric currents, which can be macroscopic currents in wires, or microscopic currents associated with electrons in atomic orbits. The magnetic field is defined in terms of force on moving charge in the Lorentz force law. Magnetic field sources are essentially dipolar in nature, having a north and south magnetic pole.

As noted, in the device 10 herein disclosed the magnets 16 or other means to generate the magnetic field 30 are shaped and/or positioned as a means to generate the magnetic field 30 sufficient to, and with the proper field lines, to yield a magnetic force vector sufficient to hold the needle 12 upright and in equilibrium between the lines of the field in relation to the top surface 15 of the base 18. Preferably of course, the magnetic field 30 generated and force and torque vectors therefrom will also rotate a non-upright needle 12 which is magnetized or becomes magnetized by the magnetic field and is deposited on the top surface 15 to the upright position, much like the needle of a rotationally engaged compass is rotated to north by force and direction of the lines in the earth's magnetic field. While this can be done on a planar version of the base 18, dimensioning the top surface 15 of the base 18 with a shaped portion such as a ramp or a concave top surface or other mechanical means to urge the needle upright works especially well.

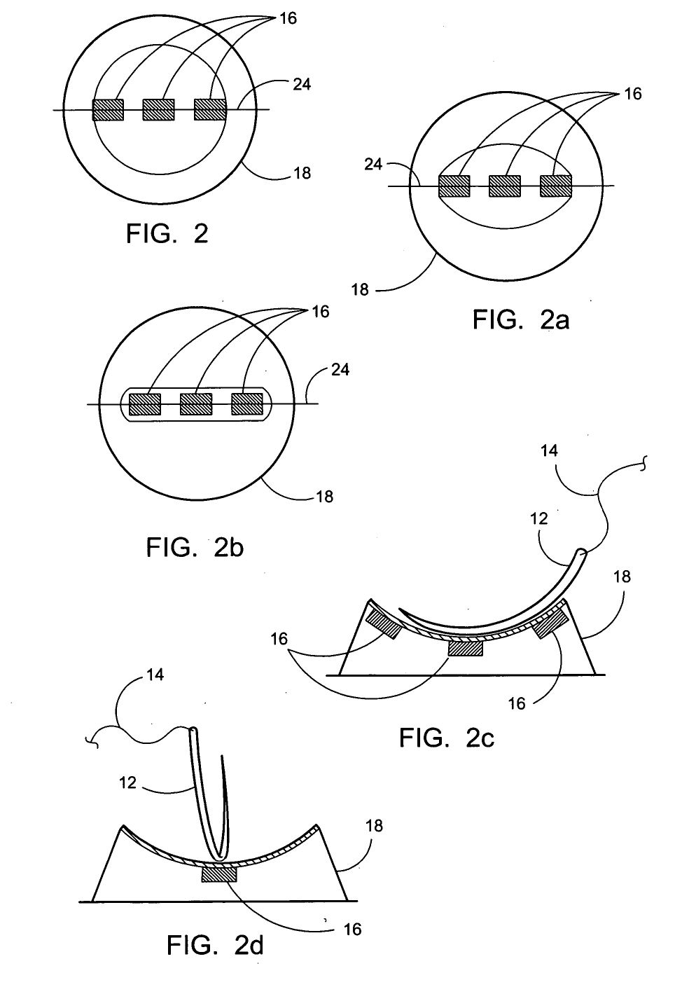

FIG. 2 depicts a top view of another preferred configuration of the device 10 into which a plurality of bar magnets 16 have been engaged to emanate the proper magnetic field above the top surface 15 of the base to yield the proper force at the correct vector to hold the needle 12 dropped or deposited thereon upright after being released. Also shown, is the magnetically generated needle axis 24 along the force vector from the magnetic field. As noted the needle axis generated above the top surface 15 is controlled by the interactions of lines of the magnetic field 30 and field orientation of the magnet or magnets 16 engaged within or below the base 18. The magnet 16 or magnets 16 can be arranged on a case by case basis depending on the shape of the top surface 15 and the shape of the magnet or magnets used with the ultimate outcome being that a needle is held upright when deposited on the top surface 15 and urged upright if dropped thereon at an angle. The overriding goal in forming the device 10 is of course to provide a magnetic field properly shaped and vectored to hold the curved needle upright on the top surface 15 once released, and also to rotate a curved needle upright if dropped on the top surface in a sideways position. The cut away views of FIGS. 2c and 2d illustrate the arranging of magnets to achieve that goal and those skilled in the art will no doubt realize that many more arrangements might be employed to yield the magnetic field with sufficient force and vectoring to hold the needle upright and all are anticipated in the scope of this invention.

In addition to the magnetic field 30, additional means to coax the needle 12 to rotate upright when dropped on the top surface 15 may be provided by forming a shaped portion of the top surface 15 by adjusting the dimensional and shape characteristics. To that end, the top surface 15 can be shaped in bowl or inverted hemispherical shape such as shown in FIG. 1, or an elongated curved shape, or a declining ramp 17 can be formed in the top surface 15 such that a deposited needle 12 will tend to slide down the deviation in the top surface 15 and toward the magnetic field 30. By shaping the top surface properly the needle will be coaxed upright by both gravity and mechanical urging of the top surface and the magnetic field. The ramp 17 as shown is narrower at the bottom end than the top which experimentation has shown tends to rotate the needle to raise up the end to be grasped at the same time as rotating the needle to an upright position. Correctly generated by means for generation of a magnetic field, the force vector of the lines forming the magnetic field 30 will be along the needle axis 24 and will hold the needle substantially upright position in relation to the landing area 28. This combined with shaping the upper surface 15 to urge a needle 12 deposited thereon to rotate upright and to expose the grasping end concurrently with the magnetic field 30 coaxing the needle 12 upright and holding it ensures easy deposit of the needle 12 on the top surface 15 and removal therefrom by easily gripping the needle 12 once turned upright.

FIG. 2a is a top view of another dimensioning of the top surface 15 of the base 18 wherein the top surface 15 has been dimensioned to an oval shaped cavity. A plurality of magnets 16 are positioned to generate the lines forming the magnetic field 30 and are engaged to the base 18 along the correct axis to urge and hold the needle upright and preferably substantially perpendicular to the top surface 15.

FIG. 2b is a top view of another configuration of the top surface 15 of the device 10 in which top surface 15 is dimensioned into the base 18 in the form of a rhombic shaped cavity. Means to generate the magnetic field 30 is provided by a plurality of magnets 16 which are positioned to generate the magnetic field 30 lines along the correct axis and at the proper force to both urge and hold the needle upright and in relation to the top surface 15 thereby allowing easy deposit on the top surface 15 and re-grasping.

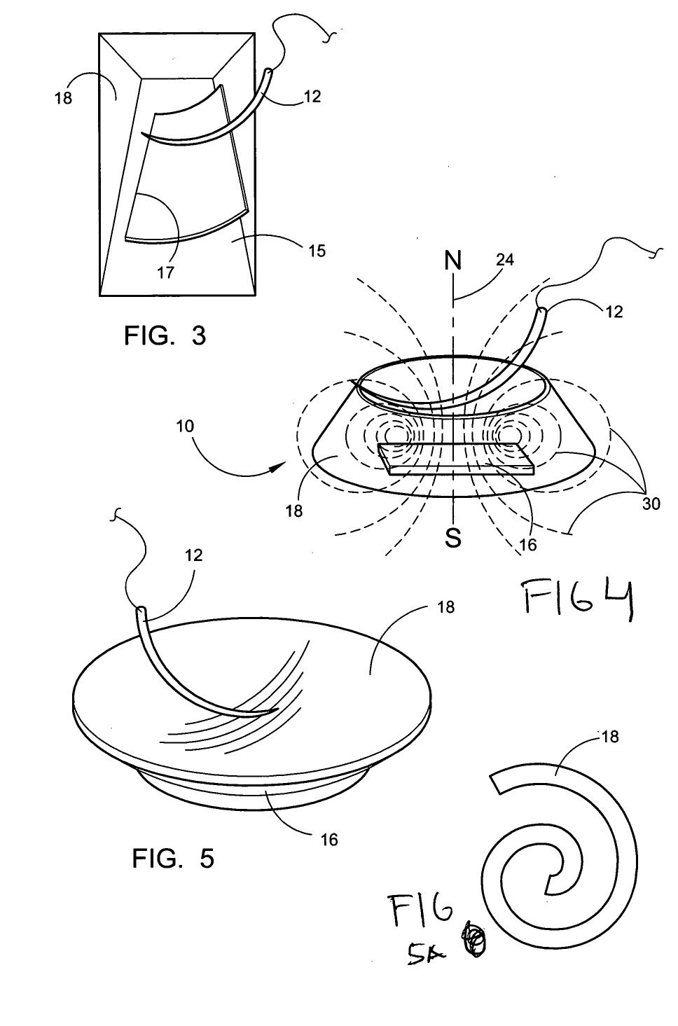

FIG. 3 depicts a top view of a dimensioning of the top surface 15 of the device 10 wherein a declining curved ramp 17 is formed in the top surface 15 which narrows from a wider point at the higher end of the ramp. The needle 12 deposited on the ramp formed in the top surface 15 will tend to slide downward toward the magnetic field along the curved path 32 forming the ramp. The combination of the declining ramp 17, the curved path 32 of the top surface 15 and the magnetic influence of the magnetic field 30 provided by the properly shaped and positioned magnets 16 under the base 18 all combine to coax the needle 12 from its position when deposited on the top surface 15 to a pickup position substantially perpendicular and in proper orientation for the next use.

FIG. 4 is a cut away view of the device 10 showing the placement of the magnet 16 or magnets 16 below the top surface 15 and the needle 12 coaxed to the upright position on the curved top surface 15. The portion of magnet 16 shown is illustrative only as the actual filed generated would be adjusted by the shape and number of magnets 16 to yield the field strength and force vector desired. Preferably the needle is coaxed to a position wherein the grasping end is from ⅓ to ½ the total length of the needle above the top surface 15 of the base 18. From this position it is easily grasped by a tool or the fingers for the next stitch in the chain. Of course the top surface 15 may also be flat or planar and with sufficient magnetic force generated with the field lines forming the correct force vector the needle 12 will remain upright on the top surface 15. However, a curved top surface 15 or top surface 15 with a ramp 17 has been found through experimentation to better urge and maintain the curved needle 12 to the upright position and to also help coax the grasping end above the rim of the curved top surface 15.

Also shown in FIG. 4 is the toroidal magnetic field 30 generated by a ring or annular shaped magnet 16 engaged with a round base 18 which has been found to be especially well adapted to coax and hold the needle 12 in the upright position. This magnetic field 30 can be envisioned as a torus shaped field enveloping the magnet 16 which is mounted below the top surface 15 of the base 18.

FIGS. 5 and 5a depicts a foldable or rollable embodiment of the device 10 wherein the base 18 is formed of flexible material as is the magnet 16. Any embodiment of the device 10 is capable of folding if the base 18 is made flexible and the magnet 16 is either flexible or sufficiently sized to allow folding or rolling of the base 18. In the case of a flexible magnet 16, such are used widely on novelty items mounted on refrigerators and for signage. The base 18 is depicted in a shape similar to that of a contact lens and provides a hemispherical or curved shaped top surface 15 when the base is unrolled. This configuration of the device 10 is especially well adapted for use inside of a closed area such as inside the body of a patient during endoscopic surgery.

In use in such a procedure, the base 18 would be rolled up and guided through an incision in the body to the inside of the body. Generally, a temporary cavity is formed in the body of the patient by the injection of carbon dioxide gas into the body to distend surrounding tissues and form a cavity of higher pressure. The device 10 would deposited in the formed cavity through an incision such as shown in FIG. 1. Thereafter, guided by video from inserted instruments, the surgeon would be able to suture the interior of the patient's body and be aided in that endeavor by the provision of the device 10 in which the needle 12 may be deposited and which will then stand upright for easy grasping for subsequent sutures. The surgeon will see the device on exterior monitors which transmit video from inside the body allowing the surgeon to handle such suturing much more easily than the conventional method of dropping the needle 12 on a body part or interior surface and then trying to grip it without injuring the patient. As noted above, however, so long as the magnet is foldable or sized correctly, any of the embodiments herein can be made flexible and those skilled in the art will no doubt realize that other configurations can be employed for a foldable or rollable device 10 that will hold a deposited needle upright with a magnetic field, and such are anticipated.

In FIG. 6 there is depicted an arrangement of a plurality of magnets 16 in varying pole positions whereby a larger magnetic field 30 is generated to coax a needle upright, especially on a concave top surface 15. Those skilled in the art will no doubt realize, that an infinite number of arrangements of magnets 16 to form a magnetic field 30 sufficient to maintain a needle 12 upright and/or coax the needle 12 upright can be formed, and all such modifications and arrangements of magnets 16 or other means to generate a magnetic field 30 as a means to hold the needle 12 upright and a means to coax a needle to an upright position are anticipated.

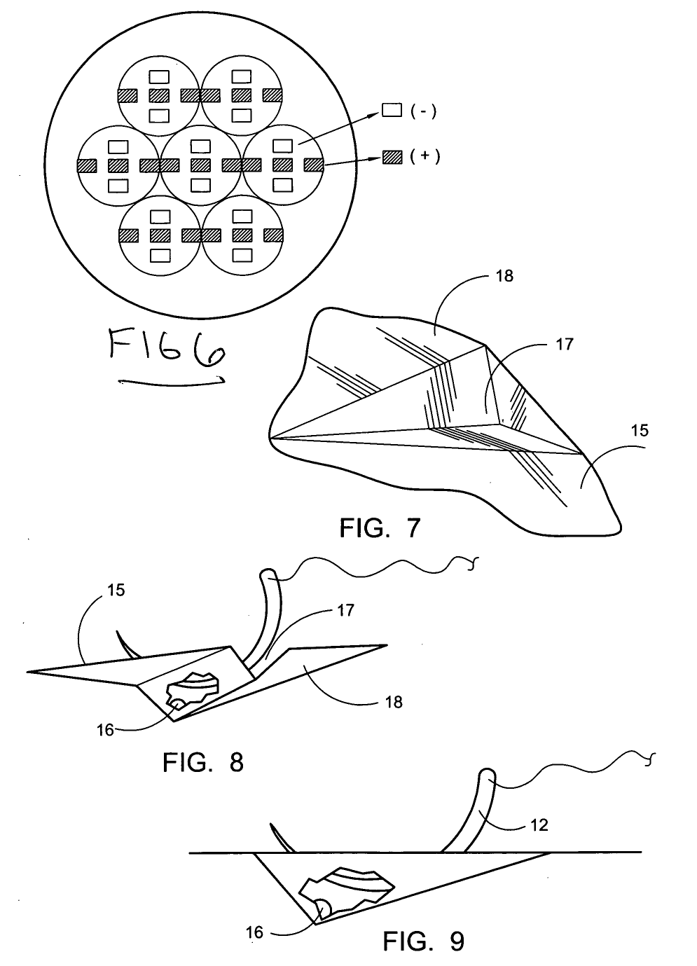

FIGS. 7-9 show an especially preferred mode of the device 10 herein disclosed and described. This mode of the device 10 features a base 18 made of flexible or resilient material having a declining ramp 17 that is V-shaped and depending from a top surface 15. The ramp 17 has two side surfaces declining to the bottom along a center line and a bottom surface along the center line that also declines from a high point adjacent to the top surface 15 to a low point. A magnet 16 is positioned adjacent to the low point. Through experimentation this embodiment of the device 10 has performed especially well in positioning the needle 12 in an upright position ready for gripping for subsequent sutures after being deposited on the top surface 15. A needle deposited or dropped over the ramp 17 on the top surface 15 will be drawn quickly to an upright position ready for subsequent gripping by the fingers or a tool.

Finally, it is envisioned that the device 10 in all embodiments and modes herein described may be widely used in surgery where a sterile environment is required. As such, the device 10 including a base 18 with the appropriate magnetic field and top surface 15 can be packaged in a sterile kit along with a curved needle 12 which is magnetized or non-magnetized as the use may require. In such a packaged kit, the user would tear open the sterile packaging and remove the base 18 and needle 12 and thereafter suture the patient using the appropriate method. In such a method, the needle 12 with appropriate thread or suturing material engaged would be gripped by a tool or the fingers for a stitch and then deposited on the top surface 15 where it would remain upright or be tilted upright in the aforementioned fashions. Thereafter the tool would be re-gripped by the fingers or a tool and the process would repeat. Of course the base 18 with the appropriate top surface 15 and magnetic field thereabove could be used with a separate needle 12; however, a sterile kit would be most convenient and allow for a throw away device 10.

Although the invention has been described with respect to particular embodiments thereof, it should be realized that various changes and modifications may be made therein without departing from the spirit and scope of the invention. While the invention as shown in the drawings and described in detail herein discloses arrangements of elements of particular construction and configuration for illustrating preferred embodiments of structure and method of operation of the present invention for rendering a needle upright on a surface using a magnetic field, it is to be understood, however, that elements of different construction and configuration and other arrangements thereof other than those illustrated and described may be employed in accordance with the spirit of this invention. Any and all such changes, alternations and modifications as would occur to those skilled in the art are considered to be within the scope of this invention as broadly defined in the appended claims.

Further, the purpose of the attached abstract is to enable the U.S. Patent and Trademark Office and the public generally, and especially the scientists, engineers and practitioners in the art who are not familiar with patent or legal terms or phraseology to determine quickly from a cursory inspection the nature and essence of the technical disclosure of the application. The abstract is neither intended to define the invention of the application, which is measured by the claims, nor is it intended to be limiting as to the scope of the invention in any way.

What is claimed is:

1. A needle positioning apparatus for positioning a curved needle: substantially upright, comprising:
a base having a top surface adapted for placement of a curved needle thereon, and a having a bottom surface;
means for generation of a magnetic field having a force vector, above said top surface; and
whereby the force vector from said magnetic field will cause said curved needle, deposited on said top surface, to remain in a substantially upright position thereon.

2. The needle postponing apparatus of claim 1 additionally having:
said curved needle deposited on said top having a pointed distal end, an eye at a second end opposite said distal end, and a curved middle portion therebetween;
said needle having a contact portion between said distal end and said middle portion of said needle; and
said top surface adapted to urge said needle to said substantially upright position with said with a contact portion of said needle engaging on said top surface, thereby positioning said eye above said top surface when in said upright position.

3. The needle postponing apparatus of claim 1 wherein:
said top surface has a shaped portion thereon positioned within said magnetic field and which is adapted to rotate said curved needle deposited thereon to said upright position, whereby a needle deposited on said top surface will be urged by said shaped area to rotate to said upright position.

4. The needle postponing apparatus of claim 2 wherein:
said top surface has a shaped portion thereon positioned within said magnetic field and which is adapted to rotate said curved needle deposited thereon to said upright position, whereby a needle deposited on said top surface will be urged by said shaped area to rotate to said upright position.

5. The needle postponing apparatus of claim 1 wherein:
said curved needle is magnetized and thereby generates a magnetic torque force to urge said needle to said upright position when said needle is deposited on said top surface within said magnetic field.

6. The needle postponing apparatus of claim 2 wherein:
said curved needle is magnetized and thereby generates a magnetic torque force to urge said needle to said upright position when said needle is deposited on said top surface within said magnetic field.

7. The needle postponing apparatus of claim 3 wherein:
said curved needle is magnetized and thereby generates a magnetic torque force to urge said needle to said upright position when said needle is deposited on said top surface within said magnetic field.

8. The needle postponing apparatus of claim 4 wherein:
said curved needle is magnetized and thereby generates a magnetic torque force to urge said needle to said upright position when said needle is deposited on said top surface within said magnetic field.

9. The needle postponing apparatus of claim 1 additionally comprising:
said base and a curved needle prepackaged in a sterile kit.

10. The needle postponing apparatus of claim 3 wherein:
said top surface is substantially planar;
said shaped portion has two angled side edges descending from said top surface and intersecting along a line defining a bottom ledge of said shaped portion; and
whereby said needle deposited on said top surface into said shaped portion will be urged by said angled side edges to rotate to said upright position.

11. The needle postponing apparatus of claim 4 wherein:
said top surface is substantially planar;
said shaped portion has two angled side edges descending from said top surface and intersecting along a line defining a bottom ledge of said shaped portion; and
whereby said needle deposited on said top surface into said shaped portion will be urged by said angled side edges to rotate to said upright position.

12. The needle postponing apparatus of claim 5 wherein:
said top surface is substantially planar;
said shaped portion has two angled side edges descending from said top surface and intersecting along a line defining a bottom ledge of said shaped portion; and
whereby said needle deposited on said top surface into said shaped portion will be urged by said angled side edges to rotate to said upright position.

13. The needle postponing apparatus of claim 7 wherein:
said top surface is substantially planar;
said shaped portion has two angled side edges descending from said top surface and intersecting along a line defining a bottom ledge of said shaped portion; and
whereby said needle deposited on said top surface into said shaped portion will be urged by said angled side edges to rotate to said upright position.

14. The needle postponing apparatus of claim 8 wherein:
said top surface is substantially planar;
said shaped portion has two angled side edges descending from said top surface and intersecting along a line defining a bottom ledge of said shaped portion; and
whereby said needle deposited on said top surface into said shaped portion will be urged by said angled side edges to rotate to said upright position.

15. The needle positioning device of claim 11 wherein:
said bottom ledge is formed at an angle descending from said top surface top surface thereby forming a slide along said bottom ledge terminating at an endwall;
a sliding of said needle deposited in said shaped portion with said distal end facing said endwall will cause a contact of said distal end of said needle with said sidewall; and
said contact of said distal end of said needle, imparting a force to said needle to rise to upright position.

16. The needle positioning device of claim 14 wherein:
said bottom ledge is formed at an angle descending from said top surface top surface thereby forming a slide along said bottom ledge terminating at an endwall;
a sliding of said needle deposited in said shaped portion with said distal end facing said endwall will cause a contact of said distal end of said needle with said sidewall; and
said contact of said distal end of said needle, imparting a force to said needle to rise to upright position.

17. The needle postponing apparatus of claim 1 wherein:
said top surface is substantially bowl shaped; and
said means to generate a magnetic field above said top surface is a ring shaped magnet.

18. The needle postponing apparatus of claim 5 wherein:
said top surface being substantially bowl shaped; and
said means to generate a magnetic field above said top surface is a ring shaped magnet.

19. The needle postponing apparatus of claim 1 wherein:
said base and said means to generate a magnetic field above said base, are both formed of a material which may be folded or rolled.

20. The needle postponing apparatus of claim 11 wherein:
said base and said means to generate a magnetic field above said base, are both formed of a material which may be folded or rolled.

21. The needle postponing apparatus of claim 14 wherein:
said base and said means to generate a magnetic field above said base, are both formed of a material which may be folded or rolled.

22. The needle postponing apparatus of claim 17 wherein:
said base and said means to generate a magnetic field above said base, are both formed of a material which may be folded or rolled.

23. The needle postponing apparatus of claim 18 wherein:
said base and said means to generate a magnetic field above said base, are both formed of a material which may be folded or rolled.

24. The needle postponing apparatus of claim 11 wherein:
said means to generate a magnetic field above said base is a magnet engaged with said base adjacent to said bottom ledge.

25. The needle postponing apparatus of claim 14 wherein:
said means to generate a magnetic field above said base is a magnet engaged with said base adjacent to said bottom ledge.

26. A method of releasing and re-grasping a curved needle for sewing using a base having a magnet generating a magnetic field above a top surface of said base, including the steps of:
positioning said base within arms reach;
placing said needle on said top surface of said base and releasing it from the grasp of the fingers or a hand held tool;
allowing said needle to be held in said generally upright on said base by said magnetic field; and
removing said needle from said base by re-grasping it with said fingers or said hand held tool.

27. A method of releasing and re-grasping a curved needle for sewing using a base having a magnet generating a magnetic field above a top surface of said base, and a top surface adapted to rotate the needle to an upright position, including the steps of:
positioning said base within arms reach;
placing said needle on said top surface of said base and releasing it from the grasp of the fingers or a hand held tool;
allowing said top surface to urge said needle to a generally upright position;
allowing said needle to be held in said generally upright on said base by said magnetic field; and
removing said needle from said base by re-grasping it with said fingers or said hand held tool.

28. The method of claim 27 including the additional steps of: for re-gripping by a tool or the fingers when released therefrom comprising:
employing a curved needle that is magnetized; and
allowing magnetic torque developed by said curved needle when deposited on said top surface to aid in rotating said curved needle to said upright position.

29. The needle postponing apparatus of claim 8 additionally comprising:
said base and a curved needle prepackaged in a sterile kit.
